(19) United States
(12) Patent Application Publication
(10) Pub. No.: US 2015/0297908 A1
(43) Pub. Date: Oct. 22, 2015

(54) METHODS AND RADIOFREQUENCY TREATMENT PROBE FOR TREATING VAGINAL LAXITY

(71) Applicant: ThermiGen, LLC, Southlake, TX (US)

(72) Inventors: Red Alinsod, Laguna Beach, CA (US); Paul R Herchman, JR., Grapevine, TX (US); Kevin D. Obrien, Coppell, TX (US)

(21) Appl. No.: 14/591,002

(22) Filed: Jan. 7, 2015

Related U.S. Application Data
(60) Provisional application No. 61/982,590, filed on Apr. 22, 2014.

(51) Int. Cl. A61N 1/40

(52) U.S. Cl. CPC A61N 1/403

(57) ABSTRACT

The invention comprises a treatment probe and method delivering radiofrequency stimulation for controlled heating of tissue within the vagina to treat vaginal laxity. The treatment probe includes a rounded disposable treatment tip to accommodate the anatomy of the vagina which is coupled with an electrode assembly, both of which are assembled to transfer radiofrequency energy to vaginal surface and subsurface. The treatment tip includes a skin temperature sensor that monitors skin temperature to maintain a pre-set treatment temperature; the entire assembly is connected to a reusable treatment probe handle and radiofrequency generator.

This application claims priority from the provisional patent application 61/982,590, filed on Apr. 22, 2014. No new material has been added.

### A. TECHNICAL FIELD OF THE INVENTION

The invention pertains to an apparatus and method of treating vaginal laxity by inducing collagen growth.

### B. BACKGROUND OF THE INVENTION

Vaginal laxity, also referred to as pelvic organ prolapse (“POP”), is defined as a condition in which vaginal wall support is lost, and in more severe cases, various pelvic organs prolapse into the vagina. This can have a serious impact on a woman's quality of life, self-esteem, and sexual enjoyment. Furthermore, vaginal laxity may contribute to urinary stress incontinence.

Vaginal laxity can be caused by a change within the collagen composition, thereby causing a weaker pelvic floor. Additionally, vaginal laxity may result from the traumatic expansion of the vaginal wall during vaginal-pregnancy.

A histological sample of the vaginal wall is depicted in FIG. 4. The vagina is lined by Stratified Squamous Epithelium 90 that features a small degree of keratinization on its Surface 95. Below the epithelium is a thick layer of dense Connective Submucosa Tissue 93, like that in the dermis of the skin. A layer of loose connective tissue containing many blood vessels and nerves follows this.

Connective tissue aberrations or deformities have been identified as a predominant cause of vaginal laxity, as the connective tissues provides critical pelvic floor support. Briefly, connective tissue is composed of collagen, elastin, smooth muscles, and microfibers. Studies have reported connective tissue defects predispose women to POP, and in turn, vaginal laxity. Women diagnosed with POP have demonstrated less total collagen content in the supportive pubocervical fascia, along with a weaker collagen matrix. Evaluation of vaginal tissue from women with and without prolapse has shown changes in expression of collagen type and elastin metabolism, an indication vaginal tissue remodels in response to POP-induced mechanical stresses. Additionally, age and neuroendocrine changes disintegrate connective tissue strength, which in turn, predisposes patients to vaginal laxity.

At present, there are two ways of dealing with POP. The first method for treatment involves surgery, which can be vaginal or abdominal. Surgical intervention is typically undesirable due to cost, pain and suffering to the patient and the possibility that even surgery will fail to be effective. Efforts to avoid surgical procedures have resulted in the development of a number of non-surgical vaginal devices, inserted into the vagina by the surgeon or the patient. Therefore, the second method of treatment requires the use of vaginal devices (pessaries) that are inserted into the vagina and mechanically reduce the prolapse by pushing the vaginal walls aside and upwards. Vaginal devices are well known for their tremendous diversity in shapes and sizes.

Some of these devices tend to block all flow of urine from the bladder. Therefore, when a patient needs to urinate, the device must be removed from the vagina or must be collapsed to remove the pressure applied against the bladder neck. Trying to solve this problem, vaginal devices were developed in special shapes that do not completely block the bladder neck, so that the patient may urinate with the device in place. These devices, however, are generally large and intrusive and, therefore, are uncomfortable to insert, wear and remove, with low patient satisfaction and compliance. They are also relatively expensive, and therefore designed to be reusable. Various pessary devices have been designed to treat prolapse in women, for example, U.S. Pat. No. 6,189,535; U.S. Pat. No. 6,158,435; U.S. Pat. No. 5,894,842; U.S. Pat. No. 5,771,899; U.S. Pat. No. 5,611,768; U.S. Pat. No. 4,823,814; and GB 19124034, the disclosures of which are herein incorporated by reference.

To overcome limitations of the afore-mentioned devices and methods, the industry needs a method to promote connective tissue remodeling, and reinforce collagen strength to avoid the use of pessaries by reinforcing vaginal anatomy and correcting vaginal laxity.

### C. SUMMARY OF THE INVENTION

The present invention provides a treatment probe and method for treating vaginal laxity. The embodiment consists of a treatment probe comprised of a curved and rounded disposable treatment tip designed to accommodate vaginal anatomy. Electrode assembly is positioned along the treatment tip to transfer radiofrequency energy to specific vaginal structures. The electrode assembly is coupled with temperature monitoring and regulatory features to maintain a predetermined temperature. A reusable radiofrequency handle connects the treatment tip to the radiofrequency generator. A protective apparatus covers the reusable treatment handle to prevent contact with the patient. The treatment probes design accommodates a specific treatment methodology to maximize treatment efficacy and optimize patient safety features, which includes monitoring and maintenance of treatment temperature.

These and other objects, features, and advantages of the present invention will become more apparent to one ordinarily skilled in the art from a review of the following Detailed Description and Claims when read in light of the accompanying drawing Figure.

### D. BRIEF DESCRIPTION OF THE SEVERAL VIEWS OF THE DRAWINGS

FIG. 1 is an exploded view of one embodiment of the invention.

FIG. 2 shows the control scheme of the invention as currently embodied.

FIG. 3A and FIG. 3B are to and side views of a current to embodiment of the disposable Interior Portion 1.

FIG. 4 shows a section of vaginal wall for explanatory purposes.

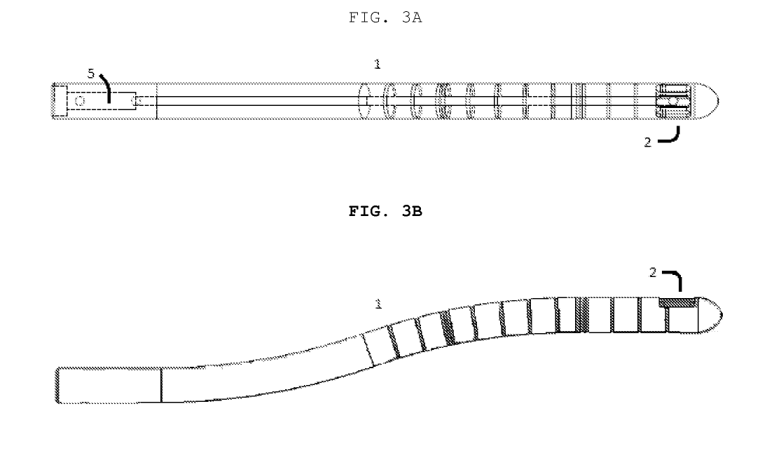

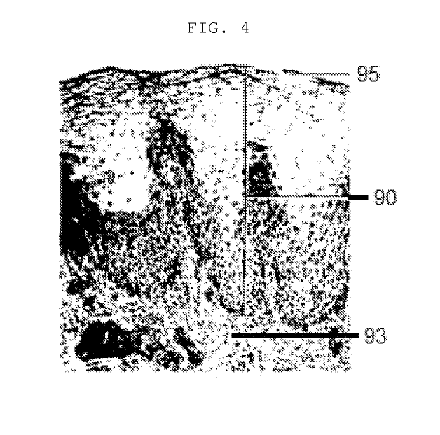

Note that FIGS. 3A and 3B were not part of the provisional patent, but contains no new material; the device was fully described as being curved to accommodate vaginal anatomy in claim 1.

### A. DETAILED DESCRIPTION OF THE INVENTION

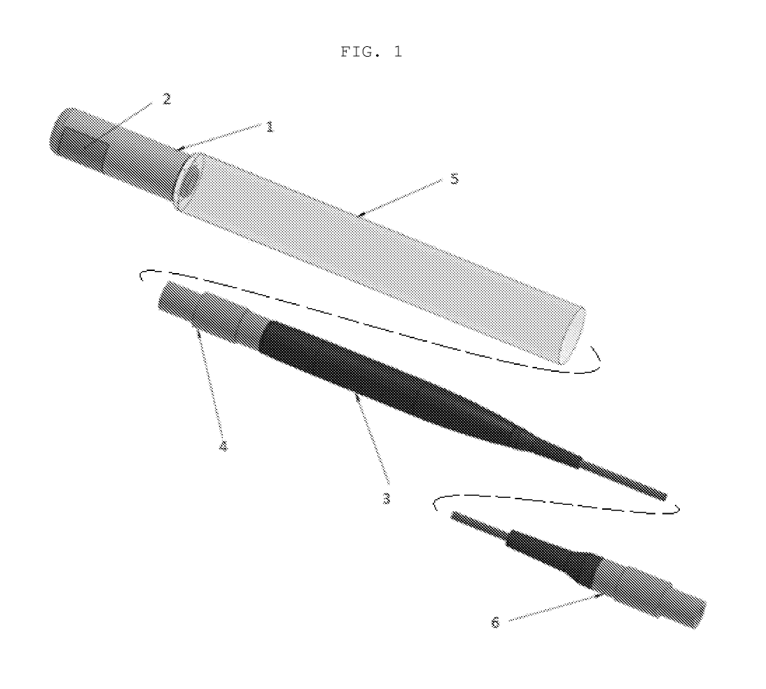

As shown in FIG. 1, one embodiment of the invention comprises a disposable Interior Portion 1 which includes an active Treatment Probe 2 and Connector 5. The Connector allows connection between the Interior Portion 1 and the radiofrequency Electrode Assembly 3. The Electrode Assembly 3 includes a to reusable RF Connection Handle 4 that attaches to the Connector 5 and an RF Source 6 coming from a connector to radiofrequency generator.

It should be noted that the names of these devices vary slightly from the provisional patent upon which this application depends, but the inventors assert that there is no new material disclosure.

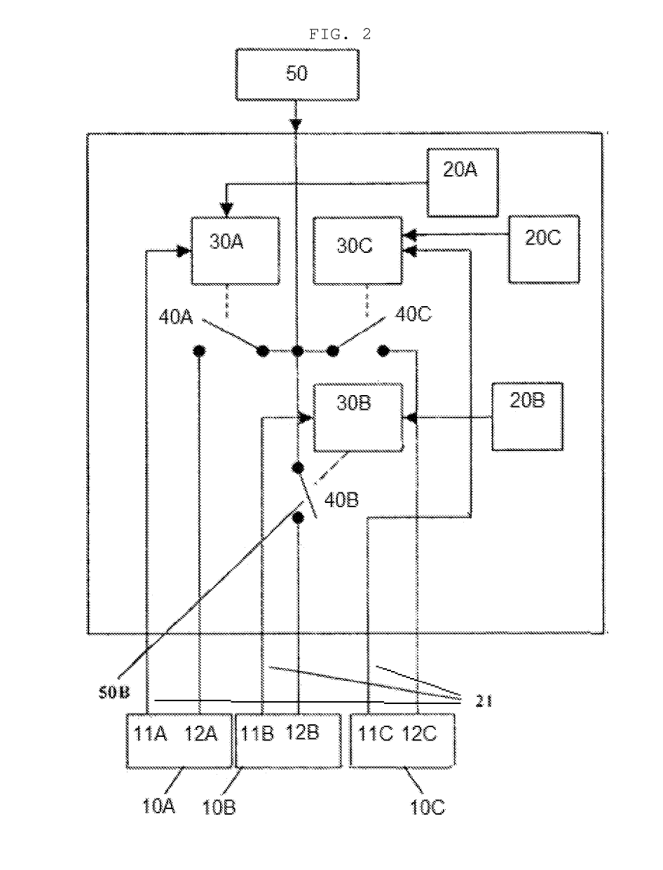

FIG. 2 is a detailed schematic illustrating an exemplary setup for how the electrode set temperature is maintained during treatment phase. As an illustration of how the circuit functions, the disclosed embodiment has three electrodes (identified in FIG. 2 by 10A, 10B and 10C). It should be understood that this embodiment permits temperature control of more than one electrode. In other words two, three, four, or more different electrodes can be controlled with this device.

As seen in FIG. 2, each Electrode 10A 10B 10C (hereafter “10”) includes an incorporated Temperature Sensor 11A 11B 11C (hereafter “11”) into the electrode that reports the temperature at the electrode tip, as well as a means, typically an industry available RF Tip 12A 12B 12C (hereafter “12”), for applying the high frequency energy to the Electrodes 10. Temperature is reported through sensor Lines 21 from each electrode to each Control Unit 30A 30B 30XC (hereafter “30”). Each Control Unit 30 also has an Input Set Temperature 20A 20B 20C (hereafter “20”), and compares the Input Set Temperature 20 and temperature reported by Sensor 11, and by comparison, determines whether to open or close Switch 40A 40B 40C (hereafter “40”). In the current embodiment, the switch is electrical, but is not limited to that construction—such control mechanisms can also be mechanical, optical, or any number of other control mechanisms known in the industry.

The Radio Frequency energy Input 50 is therefore connected and disconnected to each Electrode 10 via a Switch 40 that opens and closes as operated by its respective Control Unit 30, which is constantly comparing the user Set Temperature 20 to the temperature reported at the electrode tip by its respective Temperature Sensor 11, delivering additional RF energy to the Electrode 10. This feedback circuit will maintain Electrode 10 temperature at the user Input Set Temperature 20.

FIG. 3A and FIG. 3B are top and side views of a one-electrode embodiment, demonstrating the “curved” nature of the device, which mirrors vaginal anatomy.

In another embodiment of the present invention, an RF device includes a support structure and an RF electrode with a conductive portion and a back surface. A back plate is positioned at a proximal portion of the support structure. The back plate includes a front surface that faces the opposing back surface of the RF electrode and an opposing back surface.

In another embodiment of the present invention, an RF device includes a support structure coupled to the RF electrode. The RF electrode includes a conductive dielectric portion and a back surface. A thermoelectric cooler is coupled to the support structure. At least one sensor is coupled to the RF electrode.

In another embodiment of the present invention, an RF device includes a support structure coupled to an RF electrode. The RF electrode has conductive and dielectric portions. A thermocouple integrated with a thermistor is coupled with the RF electrode.

In another embodiment of the present invention, an RF device includes a support structure coupled to an RF electrode. The RF electrode includes conductive and dielectric portions. The RF electrode has a tissue interface surface and an opposing to back surface. A thermocouple integrated with a thermistor provides temperature monitoring and regulation along tissue interface surface.

In another embodiment of the present invention, tissue surface temperatures are maintained at a predetermined threshold using a thermocouple and optionally a thermistor to automatically turn off electrodes when the skin temperature exceeds a desirable temperature.

In another embodiment of the present invention, tissue surface temperature sensing apparatus is optimized using a treatment methodology involving manual movement of the treatment probe not exceeding one second per centimeter.

In another embodiment of the present invention, a reusable treatment handle is encased within a disposable protective apparatus composed of patient-safe materials.

In another embodiment of the present invention, a user provides a treatment methodology to optimize treatment efficacy to provide comprehensive vaginal collagen remodeling.

The inventor claims:

1. A treatment probe comprising:
a. a curved, rounded disposable treatment tip designed to accommodate vaginal anatomy;
b. an electrode assembly coupled to said treatment tip to transfer radiofrequency energy to specific vaginal structures;
c. a temperature measuring feature coupled with electrode assembly to monitor and regulate electrode and skin temperature;
d. a reusable radiofrequency handle that connects to said treatment tip;
e. a connector that connects reusable handle with the radiofrequency generator; and,
f. a disposable protective apparatus to protect reusable treatment handle.

2. The treatment probe of claim 1, wherein said electrode assembly includes a conductive portion and a dielectric portion, the RF electrode configured to capacitively couple RF energy with the tissue when at least a portion of the RF electrode is in contact with a skin surface, the conductive portion having voids, and the dielectric portion adapted to be positioned between the conductive portion and the skin surface when the RF electrode is positioned at the skin surface; and a flex circuit coupled to RF electrode.

3. The treatment probe claim 2, wherein electrode comprises a back plate coupled to support structure; and a plurality of electrical contact pads coupled to back plate.

4. The treatment probe claim 2, wherein the support structure includes first and second engagement members that provide engagement and disengagement with a hand-piece support structure.

5. The treatment probe claim 1, wherein temperature measuring feature is positioned along the back surface of the RF electrode

6. The treatment probe claim 5, wherein the temperature measuring feature detects temperature of the back surface of RF electrode

7. The treatment probe claim 1, wherein the temperature-measuring feature uses a thermocouple integrated with a thermistor.

8. The treatment probe disclosed in claim 7, wherein the thermocouple is integrated with a thermistor that uses a feedback mechanism integrated with a CPU to turn off the RF electrodes when temperatures exceed preset temperatures.

9. The treatment probe claim 8, wherein the efficacy of the thermocouple integrated with a thermistor system is most effective with manual movement of the treatment probe not exceeding 1.0 seconds per centimeter.

10. The treatment probe claim 1, wherein the protective apparatus protecting the reusable treatment handle is composed of flexible latex or solid acrylic material.

11. The treatment probe claim 1, wherein the treatment probe is placed flush along specific vaginal tissues to deliver radiofrequency energy to elevate tissue temperature.

12. The treatment probe claim 11, wherein the treatment probe is placed along the introitus, labia majora, and vaginal wall for predetermined treatment time and temperature

13. The treatment probe claim 12, wherein the treatment probe is positioned above treatment target for a specific time to reach therapeutic temperatures without compromising the skin.

14. The treatment probe claim 13, wherein the treatment probe moves in a steady-paced manner from the 11:00 o'clock position to the 1:00 o'clock position along the introitus and vaginal wall to effectively provide complete vaginal contracture without compromising the urethra.

15. The treatment probe claim 14, wherein the treatment probe is rounded and curved to provide smooth steady-paced movement, which provides improved patient comfort.
